(19) United States
(12) Patent Application Publication
(10) Pub. No.: US 2017/0128916 A1
(43) Pub. Date: May 11, 2017

(54) OXIDIC COMPOSITION

(71) Applicant: BASF SE, Ludwigshafen am Rhein (DE)

(72) Inventors: Michael LEJKOWSKI, Neckargemuend (DE); Yong LIU, Shanghai (CN); Marco HARTMANN, Jockgrim (DE); Till Christian BRUEGGEMANN, Ludwigshafen (DE); Lukas SCHULZ, Mannheim (DE); Johannes LIEBERKNECHT, Limburgerhof (DE); Armin LANGE DE OLIVEIRA, Heidelberg/Rohrbach (DE); Stephan A. SCHUNK, Heidelberg-Rohrbach (DE); Andrei-Nicolae PARVULESCU, Ruppertsberg (DE); Martin DIETERLE, Ludwigshafen (DE); Nicolai Tonio WOERZ, Darmstadt (DE); Rolf TOMPERS, Mannheim (DE); Robert MUELLER, Nussloch (DE)

(73) Assignee: BASF SE, Ludwigshafen am Rhein (DE)

(21) Appl. No.: 15/348,217

(22) Filed: Nov. 10, 2016

Related U.S. Application Data
(60) Provisional application No. 62/253,704, filed on Nov. 11, 2015.

(30) Foreign Application Priority Data
Nov. 11, 2015 (DE) 10 2015 222 198.9

(51) Int. Cl. B01J 27/199; C07C 51/353; B01J 37/08; B01J 35/00; B01J 37/02

(52) U.S. Cl. CPC B01J 27/199; B01J 35/0006; B01J 37/024; B01J 37/0236; B01J 37/086; B01J 37/088; C07C 51/353

(57) ABSTRACT

An oxidic composition comprising vanadium, tungsten, phosphorus, oxygen and optionally tin, where the molar ratio of phosphorus to the sum total of vanadium, tungsten and any tin in the oxidic composition is in the range from 1.4:1 to 2.4:1.

The present invention relates to an oxidic composition comprising vanadium, tungsten, phosphorus and oxygen, and to a process for producing the oxidic composition. The invention further relates to a process for preparing acrylic acid from acetic acid and formaldehyde using the oxidic composition.

Acrylic acid, an important monomer for production of homo- and copolymers, is typically obtained by a heterogeneously catalyzed two-stage partial oxidation proceeding from propene, with acrolein as intermediate.

A possible alternative is the preparation of acrylic acid in a heterogeneously catalyzed gas phase reaction by a condensation of formaldehyde and acetic acid. In such an aldol condensation, the catalysts used play an important role.

U.S. Pat. No. 4,165,438 A describes a process for the preparation of acrylic acid via aldol condensation from formaldehyde and acetic acid using a catalyst comprising a vanadium orthophosphate.

WO 2012/154396 A1 discloses a catalyst and the use thereof for the preparation of acrylic acid via aldol condensation from formaldehyde and acetic acid. The catalyst comprises vanadium and titanium and an oxidic additive. The oxidic additive may be Al₂O₃, ZrO₂ or SiO₂. Optionally, phosphorus and oxygen may be present in the catalyst.

WO 2013/137935 A1 discloses a process for the preparation of acrylic acid and a catalyst therefor, comprising vanadium, titanium and tungsten.

WO002013137936A1 discloses a process for the preparation of acrylic acid and a catalyst therefor, comprising vanadium, bismuth and tungsten.

The known catalysts based on oxidic compositions comprising vanadium, when used as aldol condensation catalysts in the preparation of acrylic acid, lead to formation of high amounts of carbon oxides (COₓ), meaning that they have a high selectivity for COₓ, which adversely affects the process economics.

It was therefore an object of the present invention to provide an improved catalyst for the aldol condensation of formaldehyde and acetic acid, having a reduced selectivity with respect to the formation of COₓ.

It has been found that, surprisingly, such an improved catalyst can be provided when an oxidic composition which comprises vanadium, tungsten, phosphorus, oxygen and optionally tin and which has a specific molar ratio of phosphorus to the sum total of vanadium, tungsten and any tin is used.

The present invention therefore relates to an oxidic composition comprising vanadium, tungsten, phosphorus, oxygen and optionally tin, wherein the molar ratio of phosphorus to the sum total of vanadium, tungsten and any tin in the oxidic composition is in the range from 1.4:1 to 2.4:1.

The use of such an oxidic composition having the defined molar ratio of phosphorus to the sum total of vanadium, tungsten and any tin as aldol condensation catalyst in the conversion of formaldehyde and acetic acid to acrylic acid can lower the selectivity with respect to the formation of COx (S(COx)). In addition, the selectivity of acrylic acid formation and/or the carbon conversion (C) can be increased.

Preferably, the molar ratio of phosphorus to the sum total of vanadium, tungsten and any tin in the oxidic composition of the invention is in the range from 1.8:1 to 2.3:1.

With regard to the molar ratio of vanadium to tungsten, it is preferably in the range from 10:1 to 1:100, further preferably in the range from 10:1 to 1:9, further preferably in the range from 1:1 to 9:1.

Preferably, the molar ratio of oxygen to the sum total of vanadium, tungsten and any tin is in the range from 20:1 to 1:20.

The molar ratio of oxygen to phosphorus is preferably in the range from 20:1 to 1:20.

The literature emphasizes that the addition of elements such as molybdenum, bismuth or titanium has a positive effect on the catalyst performance in the aldol condensation for preparation of acrylic acid. It has now been found that, surprisingly, in the case of the oxidic composition of the invention, it is specifically the omission of at least one of these elements, preferably of all of these elements, that has a positive effect on the selectivity of acrylic acid formation, the formation of COₓ and/or the carbon conversion (C).

Preferably, the oxidic composition therefore comprises not more than 1000 molar ppm, preferably from 0 to 100 molar ppm, of molybdenum.

Preferably, the oxidic composition comprises not more than 1000 molar ppm, preferably from 0 to 100 molar ppm, of bismuth.

Preferably, the oxidic composition comprises not more than 1000 molar ppm, preferably from 0 to 100 molar ppm, of titanium.

In a preferred configuration, the oxidic composition comprises from 0 to 1000 molar ppm of molybdenum, from 0 to 1000 molar ppm of bismuth and from 0 to 1000 molar ppm of titanium.

In a preferred configuration, the oxidic composition comprises tin.

Preferably, in this configuration of the oxidic composition, the molar ratio of vanadium to tin is in the range from 100:1 to 1:100, preferably in the range from 10:1 to 1:10.

Preferably, at least 99% by weight, preferably at least 99.5% by weight, further preferably at least 99.9% by weight, of the oxidic composition consists of vanadium, tungsten, phosphorus, oxygen and optionally tin.

In a preferred configuration, the oxidic composition additionally comprises a support material. In this configuration, the oxidic composition is preferably in supported form on a support material.

Preferably, the support material comprises at least one semimetal oxide or at least one metal oxide or a mixture of at least one semimetal oxide and at least one metal oxide. Preferably, the support material consists of at least one semimetal oxide or at least one metal oxide or a mixture of at least one semimetal oxide and at least one metal oxide. The support material is preferably selected from the group consisting of SiO₂, Al₂O₃, ZrO₂, and a mixture of two or three thereof. Preferably, the support material comprises SiO₂.

In principle, the support material is not subject to any particular restrictions with regard to the amounts of the components thereof. Preferably, at least 95% by weight, preferably at least 98% by weight, further preferably at least 99% by weight, further preferably at least 99.5% by weight, of the support material consists of SiO₂.

Preferably, at least 95% by weight, preferably at least 98% by weight, further preferably at least 99% by weight, further preferably at least 99.5% by weight, of the oxidic composition consists of the oxidic composition as described above and the support material.

In a preferred configuration, the oxidic composition is a catalyst. Preferably, the oxidic composition is an aldol condensation catalyst.

In a first particularly preferred configuration, the oxidic composition is an unsupported catalyst. Preferably, in this configuration, the oxidic composition is an unsupported aldol condensation catalyst.

In a further particularly preferred configuration, the oxidic composition is a supported catalyst. Preferably, in this configuration, the oxidic composition is a supported aldol condensation catalyst.

Preparation of Oxidic Composition

The present invention also relates to a process for producing an oxidic composition, comprising providing a support material; providing an aqueous vanadium solution, an aqueous tungsten solution, an aqueous phosphorus solution and optionally an aqueous tin solution; impregnating the support material with the aqueous vanadium solution and the aqueous tungsten solution and optionally the aqueous tin solution; optionally drying the resulting impregnated material; impregnating the optionally dried material with the aqueous phosphorus solution; optionally drying the resulting impregnated material; calcining the optionally dried material.

In one configuration of the process, the support material is impregnated with an aqueous solution containing at least tungsten and vanadium (co-impregnation). It is also conceivable to impregnate the support material with an aqueous solution containing vanadium and/or tungsten and tin. The only conditions for such a co-impregnation are the miscibility of the individual elements and that the total volume of the mixed solution must not exceed the water absorption. The aqueous phosphorus solution is preferably applied separately, more preferably after the impregnation of the support material with vanadium, tungsten and optionally tin.

Preference is given to using the process for producing an above-described oxidic composition. Further preference is given to using the process for producing an above-described oxidic composition additionally comprising a support material. Further preference is given to using the process for producing an above-described oxidic composition which is a supported catalyst, preferably a supported aldol condensation catalyst.

Preferably, the aqueous solutions provided comprise a total of not more than 1000 molar ppm, preferably from 0 to 100 molar ppm, of molybdenum, not more than 1000 molar ppm, preferably from 0 to 100 molar ppm, of bismuth and not more than 1000 molar ppm, preferably from 0 to 100 molar ppm, of titanium.

Preferably, the support material comprises at least one semimetal oxide or at least one metal oxide or a mixture of at least one semimetal oxide and at least one metal oxide. Preferably, the support material consists of at least one semimetal oxide or at least one metal oxide or a mixture of at least one semimetal oxide and at least one metal oxide.

The support material is preferably selected from the group consisting of SiO₂, Al₂O₃, ZrO₂, and a mixture of two or three thereof. Preferably, the support material comprises SiO₂.

Preferably, at least 95% by weight, preferably at least 98% by weight, further preferably at least 99% by weight, further preferably at least 99.5% by weight, of the support material consists of SiO₂.

In principle, the aqueous vanadium solution is not subject to any particular restrictions with regard to the vanadium compound used. Preference is given to using halogen-free vanadium compounds. Preferably, the aqueous vanadium solution comprises vanadium citrate or vanadium oxalate or a mixture thereof.

In principle, the aqueous phosphorus solution is not subject to any particular restrictions with regard to the phosphorus compound used. Preferably, the aqueous phosphorus solution comprises phosphoric acid.

In principle, the aqueous tin solution is not subject to any particular restrictions with regard to the tin compound used. Preference is given to using halogen-free tin compounds. Preferably, the aqueous tin solution comprises tin oxalate, optionally as a mixture with nitric acid.

In principle, the aqueous tungsten solution is not subject to any particular restrictions with regard to the tungsten compound used. Preferably, the aqueous tungsten solution comprises ammonium metatungstate.

In a preferred configuration of the process for producing an oxidic composition, the process comprises
(i) providing the support material;
(ii) providing the aqueous vanadium solution, the aqueous tungsten solution; the aqueous phosphorus solution;
(iii) impregnating the support material with the aqueous vanadium solution;
(iv) preferably drying the material obtained in (iii);
(V) impregnating the material obtained in (iv) with the aqueous tungsten solution;
(vi) preferably drying the material obtained in (V);
(vii) impregnating the material obtained in (vi) with the aqueous phosphorus solution;
(viii) preferably drying the material obtained in (vii);
(ix) calcining the material obtained in (viii).

In a preferred configuration, the drying is effected in (iv). It is preferably effected at a temperature of the gas atmosphere used for drying in the range from 60 to 120° C., further preferably in the range from 70 to 90° C. The gas atmosphere is preferably selected from the group consisting of oxygen, nitrogen, air and lean air, and is further preferably air.

In principle, the drying in (iv) is not subject to any particular restrictions in terms of duration, provided that the drying takes place. Preference is given to conducting the drying in (iv) for a period in the range from 0.5 to 40 h, preferably in the range from 1 to 18 h.

In a further preferred configuration, the drying is effected in (vi). It is preferably effected at a temperature of the gas atmosphere used for drying in the range from 60 to 120° C., further preferably in the range from 70 to 90° C. The gas atmosphere is preferably selected from the group consisting of oxygen, nitrogen, air and lean air, and is preferably air.

In principle, the drying in (vi) is not subject to any particular restrictions in terms of duration, provided that the drying takes place. Preference is given to conducting the drying in (vi) for a period in the range from 0.5 to 40 h, preferably in the range from 1 to 18 h.

In a further preferred configuration, the drying is effected in (viii). It is preferably effected at a temperature of the gas atmosphere used for drying in the range from 60 to 120° C., further preferably in the range from 70 to 90° C. The gas atmosphere is preferably selected from the group consisting of oxygen, nitrogen, air and lean air, and is preferably air.

In principle, the drying in (viii) is not subject to any particular restrictions in terms of duration, provided that the drying takes place. Preference is given to conducting the drying in (viii) for a period in the range from 0.5 to 40 h, preferably in the range from 1 to 18 h.

In a preferred configuration of the process, the process consists of steps (i) to (ix).

With regard to the preferred configuration of the process comprising steps (i) to (ix), preferably consisting of steps (i) to (ix), preference is given to
(ii) additionally providing an aqueous tin solution and to the process additionally comprising
(a) impregnating the material obtained in (iv) or that obtained in (vii) with the aqueous tin solution;
(b) preferably drying the material obtained in (a),
where (a) to (b) preferably follow (iv) and precede (V) or follow (vi) and precede (vii).

Preference is given to effecting the drying in (b). It is preferably effected at a temperature of the gas atmosphere used for drying in the range from 60 to 120° C., further preferably in the range from 70 to 90° C. The gas atmosphere is preferably selected from the group consisting of oxygen, nitrogen, air and lean air, and is preferably air.

In principle, the drying in (b) is not subject to any particular restrictions in terms of duration, provided that the drying takes place. Preference is given to conducting the drying in (b) for a period in the range from 0.5 to 40 h, preferably in the range from 1 to 18 h.

Preferably, the process consists of steps (i) to (ix) and (a) to (b).

In principle, the calcining is not subject to any particular restrictions with regard to the temperature. Preferably, the calcining is conducted at a temperature of the gas atmosphere used for drying in the range from 200 to 500° C., preferably in the range from 240 to 480° C., further preferably in the range from 240 to 280° C.

In principle, the calcining is not subject to any particular restrictions with regard to the duration. Preferably, the calcining is conducted for a duration in the range from 1 to 10 h, preferably in the range from 1 to 8 h, further preferably in the range from 1 to 3 h.

Preferably, the calcining is effected with a heating ramp of 0.5 K/min to 5 K/minute, preferably from 0.5 K/min to 2 K/min.

The present invention likewise relates to an oxidic composition, preferably a catalyst, further preferably an aldol condensation catalyst, obtained or obtainable by the above-described process. Further preferably, the present invention likewise relates to an oxidic composition, preferably a catalyst, further preferably an aldol condensation catalyst, obtained or obtainable by the process comprising (i) to (ix), and additionally comprising (a) to (b). Further preferably, the present invention relates to an oxidic composition, preferably a catalyst, further preferably an aldol condensation catalyst, obtained or obtainable by the process consisting of (i) to (ix) and (a) to (b).

The present invention likewise relates to the use of the above-described oxidic composition as catalyst, preferably as aldol condensation catalyst, further preferably as aldol condensation catalyst for preparation of acrylic acid from acetic acid and formaldehyde.

Preparation of Acrylic Acid from Acetic Acid and Formaldehyde

The present invention further relates to a process for preparing acrylic acid from acetic acid and formaldehyde, comprising
(i) providing a stream S1 comprising acetic acid and formaldehyde;
(ii) contacting stream S1 with an aldol condensation catalyst comprising, preferably consisting of, an oxidic composition comprising vanadium, tungsten, phosphorus, oxygen and optionally tin, where the molar ratio of phosphorus to the sum total of vanadium, tungsten and any tin in the oxidic composition is in the range from 1.4:1 to 2.4:1, as described in detail above, to obtain a stream S2 comprising acrylic acid.

In principle, stream S1 is not restricted in terms of the molar ratio of formaldehyde:acetic acid. Preferably, the molar ratio of acetic acid:formaldehyde in stream S1 in (i) is not less than 0.25:1.

Preferably, the molar ratio of acetic acid:formaldehyde in stream S1 in (i) is not more than 4.4:1.

Preferably, the molar ratio of acetic acid:formaldehyde in stream S1 in (i) is in the range from 0.25:1 to 4.4:1, preferably in the range from 0.5:1 to 2:1, further preferably in the range from 0.8:1 to 1.2:1.

Useful sources for the acetic acid in principle include any suitable source comprising at least a proportion of acetic acid. This may be acetic acid fed fresh to the process. It may likewise be acetic acid which has not been converted in the above-described process and which, for example after removal from the product stream in one or more workup steps, is recycled into the process. A combination of acetic acid fed fresh to the process and acetic acid recycled into the process is likewise possible. It is likewise possible to use acetic acid adducts, for example acetic anhydride.

Useful sources for formaldehyde likewise in principle include any suitable source comprising at least a proportion of formaldehyde. This may be formaldehyde fed fresh to the process. It may likewise be formaldehyde which has not been converted in the above-described process and which, for example after removal from the product stream in one or more workup steps, is recycled into the process. A combination of formaldehyde fed fresh to the process and formaldehyde recycled into the process is likewise possible. For example, the source used for the formaldehyde may be an aqueous formaldehyde solution (formalin). It is likewise possible to use a formaldehyde source which affords formaldehyde, for instance trioxane or paraformaldehyde. Preferably, the source used for the formaldehyde is an aqueous formaldehyde solution. Preferably, the aqueous formaldehyde solution has a formaldehyde content in the range from 20% to 85% by weight, preferably from 30% to 80% by weight, further preferably from 40% to 60% by weight.

It is conceivable in principle that stream S1 in (i) consists of formaldehyde and acetic acid.

Preferably, stream S1 comprises at least one further component in addition to formaldehyde and acetic acid, and stream S1 in (i) further preferably additionally comprises at least one of the components water, inert gas and oxygen.

Preferably, stream S1 in (i) additionally comprises inert gas.

In principle, stream S1 is not subject to any particular restrictions in terms of the inert gas content. Preferably, the inert gas content of stream S1 in (i) is in the range from 0.1% to 85.0% by volume, preferably in the range from 40% to 75% by volume, further preferably in the range from 50% to 70% by volume, based on the total volume of stream S1.

In the context of the present invention, inert gas shall be all the materials that are gaseous under the process conditions selected in each case and are inert in stage (i). “Inert” in this context means that the gaseous material in a single pass through the reaction zone is converted to an extent of less than 5 mol%, preferably to an extent of less than 2 mol%, more preferably to an extent of less than 1 mol%. Regardless of this definition, water, oxygen, carbon dioxide, carbon monoxide, propionic acid, formic acid, methanol, methyl acetate, acetaldehyde, methyl acrylate, ethene, acetone, methyl formate and acrylic acid shall not be covered by the term “inert gas”. In this context, the term “inert gas” as used in the context of the present invention refers either to a single gas or to a mixture of two or more gases. For example, useful inert gases include helium, neon, argon, krypton, radon, xenon, nitrogen, sulfur hexafluoride and gas mixtures of two or more thereof.

Preferably, the inert gas in stream S1 in (i) comprises nitrogen, there being no restrictions in principle with regard to the proportion of nitrogen. Preferably, at least 95% by weight, further preferably at least 98% by weight, further preferably at least 99% by weight, of the inert gas consists of nitrogen.

Preferably, stream S1 in (i) additionally comprises water and oxygen. Preferably, at least 65% by volume and preferably at least 80% by volume of stream S1 in (i) consists of formaldehyde, acetic acid, water, oxygen and inert gas.

Preferably, stream S1 in (i) additionally comprises one or more of the compounds carbon dioxide, carbon monoxide, propionic acid, formic acid, methanol, methyl acetate, acetaldehyde, methyl acrylate, ethene, acetone, methyl formate and acrylic acid.

Preferably, stream S1 in (i) is gaseous.

Contacting of Stream S1 with an Aldol Condensation Catalyst in (ii)

In (ii), stream S1 is contacted with an aldol condensation catalyst to obtain a gaseous stream S2 comprising acrylic acid.

The contacting is preferably continuous.

Preferably, the contacting in (ii) is effected in at least one reactor, preferably in at least two reactors, further preferably in at least two reactors connected in parallel, which are preferably operaced in alternation, the reactors preferably being fixed bed reactors. In the alternating mode of operation, at least one reactor is always in operation. The fixed bed reactors are configured, for example, as shell and tube reactors or thermoplate reactors. In the case of a shell and tube reactor, the catalytically active fixed bed is advantageously within the catalyst tubes, with fluid heat carrier flowing around them.

The catalyst hourly space velocity with regard to the contacting in (ii) in the reactor is preferably chosen such that a balanced ratio of the parameters of conversion, selectivity, space-time yield, reactor geometry and reactor dimensions can be achieved.

Preferably, the contacting in (ii) in a fixed bed reactor is effected at a catalyst hourly space velocity in the range from 0.01 to 50 kg/(h*kg), preferably in the range from 0.1 to 40 kg/(h*kg), further preferably in the range from 0.5 to 30 kg/(h*kg), the catalyst hourly space velocity being defined as the mass of stream S1 in kg per hour and per unit mass of aldol condensation catalyst in kg.

The contacting in (ii) in the reactor is not subject to any particular restrictions with regard to the pressure, provided that the contacting of stream S1 with the aldol condensation catalyst gives a stream S2 comprising acrylic acid.

Preferably, the contacting in (ii) in a fixed bed reactor is effected at an absolute pressure in the range from 0.5 to 5 bar, further preferably in the range from 0.8 to 3 bar, further preferably in the range from 1 to 1.8 bar.

Stream S1 may in principle be fed to the reaction zone at any temperature suitable for the process of the invention. Preferably, stream S1 is fed to the reaction zone at a temperature at which it is entirely in gaseous form. Further preferably, stream S1 is fed to the reaction zone at a temperature in the range from 150 to 450° C., further preferably from 200 to 400° C., further preferably from 250 to 390° C.

Preferably, stream S2 obtained in (ii) is at a temperature in the range from 200 to 450° C., preferably in the range from 250 to 400° C., further preferably in the range from 300 to 400° C.

Preferably, the ratio of the volume of acrylic acid to the sum total of the volumes of formaldehyde and acetic acid in stream S2 obtained in (ii) is in the range from 0.1:1 to 2.0:1, preferably in the range from 0.4:1 to 1.2:1.

The present invention is illustrated in detail by the following embodiments and combinations of embodiments which are apparent from the corresponding dependency references and other references:
1. An oxidic composition comprising vanadium, tungsten, phosphorus, oxygen and optionally tin, wherein the molar ratio of phosphorus to the sum total of vanadium, tungsten and any tin in the oxidic composition is in the range from 1.4:1 to 2.4:1.
2. The oxidic composition according to embodiment 1, wherein the molar ratio of phosphorus to the sum total of vanadium, tungsten and any tin is in the range from 1.8:1 to 2.3:1.
3. The oxidic composition according to embodiment 1 or 2, wherein the molar ratio of vanadium to tungsten in the oxidic composition is in the range from 10:1 to 1:100, preferably in the range from 10:1 to 1:9, further preferably in the range from 1:1 to 9:1.
4. The oxidic composition according to any of embodiments 1 to 3, wherein the oxidic composition comprises not more than 1000 molar ppm, preferably from 0 to 100 molar ppm, of molybdenum.
5. The oxidic composition according to any of embodiments 1 to 4, wherein the oxidic composition comprises not more than 1000 molar ppm, preferably from 0 to 100 molar ppm, of bismuth.
6. The oxidic composition according to any of embodiments 1 to 5, wherein the oxidic composition comprises not more than 1000 molar ppm, preferably from 0 to 100 molar ppm, of titanium.
7. The oxidic composition according to any of embodiments 1 to 6, wherein the oxidic composition comprises from 0 to 1000 molar ppm of molybdenum, from 0 to 1000 molar ppm of bismuth and from 0 to 1000 molar ppm of titanium.
8. The oxidic composition according to any of embodiments 1 to 7, wherein the oxidic composition comprises tin.
9. The oxidic composition according to embodiment 8, wherein the molar ratio of vanadium to tin in the oxidic composition is in the range from 100:1 to 1:100, preferably in the range from 10:1 to 1:10.
10. The oxidic composition according to any of embodiments 1 to 9, wherein at least 99% by weight, preferably at least 99.5% by weight, further preferably at least 99.9% by weight, of the oxidic composition consists of vanadium, tungsten, phosphorus, oxygen and optionally tin.
11. The oxidic composition according to any of embodiments 1 to 10, additionally comprising a support material.
12. The oxidic composition according to any of embodiments 1 to 11, supported on a support material.
13. The oxidic composition according to embodiment 11 or 12, wherein the support material comprises at least one semimetal oxide or at least one metal oxide or a mixture of at least one semimetal oxide and at least one metal oxide, preferably consists of at least one semimetal oxide or at least one metal oxide or a mixture of at least one semimetal oxide and at least one metal oxide.
14. The oxidic composition according to any of embodiments 11 to 13, wherein the support material is selected from the group consisting of SiO₂, Al₂O₃, ZrO₂, and a mixture of two or three thereof, and wherein the support material preferably comprises SiO₂.
15. The oxidic composition according to any of embodiments 11 to 14, wherein at least 95% by weight, preferably at least 98% by weight, further preferably at least 99% by weight, further preferably at least 99.5% by weight, of the support material consists of SiO₂.
16. The oxidic composition according to any of embodiments 11 to 15, wherein at least 95% by weight, preferably at least 98% by weight, further preferably at least 99% by weight, further preferably at least 99.5% by weight, of the oxidic composition consists of the oxidic composition according to any of embodiments 1 to 10 and the support material.
17. The oxidic composition according to any of embodiments 1 to 16, wherein the oxidic composition is a catalyst, preferably an aldol condensation catalyst.
18. The oxidic composition according to any of embodiments 1 to 10, wherein the oxidic composition is an unsupported catalyst, preferably an unsupported aldol condensation catalyst.
19. The oxidic composition according to any of embodiments 11 to 16, wherein the oxidic composition is a supported catalyst, preferably a supported aldol condensation catalyst.
20. A process for producing an oxidic composition, comprising providing a support material; providing an aqueous vanadium solution, an aqueous tungsten solution, an aqueous phosphorus solution and optionally an aqueous tin solution; impregnating the support material with the aqueous vanadium solution and the aqueous tungsten solution and optionally the aqueous tin solution; optionally drying the resulting impregnated material; impregnating the optionally dried material with the aqueous phosphorus solution; optionally drying the resulting impregnated material; calcining the optionally dried material.
21. The process according to embodiment 20 for producing an oxidic composition according to any of embodiments 1 to 19, preferably according to any of embodiments 11 to 19, further preferably according to embodiment 19.
22. The process according to embodiment 20 or 21, wherein the aqueous solutions provided comprise a total of not more than 1000 molar ppm, preferably from 0 to 100 molar ppm, of molybdenum, not more than 1000 molar ppm, preferably from 0 to 100 molar ppm, of bismuth and not more than 1000 molar ppm, preferably from 0 to 100 molar ppm, of titanium.
23. The process according to any of embodiments 20 to 22, wherein the support material comprises at least one semimetal oxide or at least one metal oxide or a mixture of at least one semimetal oxide and at least one metal oxide, preferably consists of at least one semimetal oxide or at least one metal oxide or a mixture of at least one semimetal oxide and at least one metal oxide.
24. The process according to any of embodiments 20 to 23, wherein the support material is selected from the group consisting of SiO₂, Al₂O₃, ZrO₂ and a mixture of two or three thereof, and wherein the support material preferably comprises SiO₂.
25. The process according to any of embodiments 20 to 24, wherein at least 95% by weight, preferably at least 98% by weight, further preferably at least 99% by weight, further preferably at least 99.5% by weight, of the support material consists of SiO₂.
26. The process according to any of embodiments 20 to 25, wherein the aqueous vanadium solution comprises vanadium citrate or vanadium oxalate or a mixture thereof.
27. The process according to any of embodiments 20 to 26, wherein the aqueous phosphorus solution comprises phosphoric acid.
28. The process according to any of embodiments 20 to 27, wherein the aqueous tin solution comprises tin oxalate, optionally as a mixture with nitric acid.
29. The process according to any of embodiments 20 to 28, wherein the aqueous tungsten solution comprises ammonium metatungstate.
30. The process according to any of embodiments 20 to 29, comprising
   (i) providing the support material;
   (ii) providing the aqueous vanadium solution, the aqueous tungsten solution; the aqueous phosphorus solution;
   (iii) impregnating the support material with the aqueous vanadium solution;
   (iv) preferably drying the material obtained in (iii);
   (V) impregnating the material obtained in (iv) with the aqueous tungsten solution;
   (vi) preferably drying the material obtained in (V);
   (vii) impregnating the material obtained in (vi) with the aqueous phosphorus solution;
   (viii) preferably drying the material obtained in (vii);
   (ix) calcining the material obtained in (viii).
31. The process according to embodiment 30, wherein the drying is effected in (iv), preferably at a temperature of the gas atmosphere used for drying in the range from 60 to 120° C., further preferably in the range from 70 to 90° C., where the gas atmosphere is preferably selected from the group consisting of oxygen, nitrogen, air and lean air, and is preferably air.
32. The process according to embodiment 31, wherein the drying in (iv) is conducted for a period in the range from 0.5 to 40 h, preferably in the range from 1 to 18 h.
33. The process according to any of embodiments 30 to 32, wherein the drying is effected in (vi), preferably at a temperature of the gas atmosphere used for drying in the range from 60 to 120° C., further preferably in the range from 70 to 90° C., where the gas atmosphere is preferably selected from the group consisting of oxygen, nitrogen, air and lean air, and is preferably air.
34. The process according to embodiment 33, wherein the drying in (vi) is conducted for a period in the range from 0.5 to 40 h, preferably in the range from 1 to 18 h.
35. The process according to any of embodiments 30 to 34, wherein the drying is effected in (viii), preferably at a temperature of the gas atmosphere used for drying in the range from 60 to 120° C., further preferably in the range from 70 to 90° C., where the gas atmosphere is preferably selected from the group consisting of oxygen, nitrogen, air and lean air, and is preferably air.
36. The process according to embodiment 35, wherein the drying in (viii) is conducted for a period in the range from 0.5 to 40 h, preferably in the range from 1 to 18 h.
37. The process according to any of embodiments 30 to 36, wherein the process consists of steps (i) to (ix).
38. The process according to any of embodiments 30 to 36, wherein (ii) additionally comprises the providing of an aqueous tin solution and the process additionally comprises
   (a) impregnating the material obtained in (iv) or that obtained in (vii) with the aqueous tin solution;
   (b) preferably drying the material obtained in (a),
   where (a) to (b) preferably follow (iv) and precede (V) or follow (vi) and precede (vii).
39. The process according to embodiment 38, wherein the drying is effected in (b), preferably at a temperature of the gas atmosphere used for drying in the range from 60 to 120° C., further preferably in the range from 70 to 90° C., where the gas atmosphere is preferably selected from the group consisting of oxygen, nitrogen, air and lean air, and is preferably air.
40. The process according to embodiment 39, wherein the drying in (b) is conducted for a period in the range from 0.5 to 40 h, preferably in the range from 1 to 18 h.
41. The process according to any of embodiments 38 to 40, wherein the process consists of steps (i) to (ix) and (a) to (b).
42. The process according to any of embodiments 30 to 41, wherein the calcining is conducted at a temperature of the gas atmosphere used for drying in the range from 200 to 500° C., preferably in the range from 240 to 480° C., further preferably in the range from 240 to 280° C.
43. The process according to any of embodiments 30 to 42, wherein the calcining is conducted for a duration in the range from 1 to 10 h, preferably in the range from 1 to 8 h, further preferably in the range from 1 to 3 h.
44. The process according to any of embodiments 30 to 43, wherein the calcining is effected with a heating ramp of 0.5 K/min to 5 K/min, preferably from 0.5 K/min to 2 K/min.
45. An oxidic composition, preferably catalyst, further preferably aldol condensation catalyst, obtained or obtainable by a process according to any of embodiments 21 to 44, preferably according to embodiment 38 or 41.
46. The use of an oxidic composition according to any of embodiments 1 to 17 or 45 as catalyst, preferably as aldol condensation catalyst, further preferably as aldol condensation catalyst for preparation of acrylic acid from acetic acid and formaldehyde.
47. A process for preparing acrylic acid from acetic acid and formaldehyde, comprising
   (i) providing a stream S1 comprising acetic acid and formaldehyde;
   (ii) contacting stream S1 with an aldol condensation catalyst comprising, preferably consisting of, an oxidic composition according to any of embodiments 1 to 17 or 45 to obtain a stream S2 comprising acrylic acid.
48. The process according to embodiment 47, wherein the molar ratio of acetic acid:formaldehyde in stream S1 in (i) is not less than 0.25:1.
49. The process according to either of embodiments 47 and 48, wherein the molar ratio of acetic acid:formaldehyde in stream S1 in (i) is not more than 4.4:1.
50. The process according to any of embodiments 47 to 49, wherein the molar ratio of acetic acid:formaldehyde in stream S1 in (i) is in the range from 0.25:1 to 4.4:1, preferably in the range from 0.5:1 to 2:1, further preferably in the range from 0.8:1 to 1.2:1.
51. The process according to any of embodiments 47 to 50, wherein stream S1 in (i) additionally comprises inert gas.
52. The process according to embodiment 51, wherein the inert gas content of stream S1 in (i) is in the range from 0.1% to 85.0% by volume, preferably in the range from 40% to 75% by volume, further preferably in the range from 50% to 70% by volume, based on the total volume of stream S1.
53. The process according to embodiment 51 or 52, wherein the inert gas in stream S1 in (i) comprises nitrogen, where preferably at least 95% by weight, further preferably at least 98% by weight, further preferably at least 99% by weight, of the inert gas consists of nitrogen.
54. The process according to any of embodiments 47 to 53, wherein stream S1 in (i) additionally comprises water and oxygen, wherein at least 65% by volume and preferably at least 80% by volume of stream S1 in (i) consists of formaldehyde, acetic acid, water, oxygen and inert gas.
55. The process according to any of embodiments 47 to 54, wherein stream S1 in (i) additionally comprises one or more of the compounds carbon dioxide, carbon monoxide, propionic acid, formic acid, methanol, methyl acetate, acetaldehyde, methyl acrylate, ethene, acetone, methyl formate and acrylic acid.
56. The process according to any of embodiments 47 to 55, wherein stream S1 in (i) is gaseous.
57. The process according to any of embodiments 47 to 56, wherein the contacting in (ii) is effected continuously.
58. The process according to any of embodiments 47 to 57, wherein the contacting in (ii) is effected in at least one reactor, preferably at least two reactors, further preferably in at least two reactors connected in parallel, which are preferably operated in alternation, the reactors preferably being fixed bed reactors.
59. The process according to embodiment 58, wherein the contacting in (ii) in a fixed bed reactor is effected at a catalyst hourly space velocity in the range from 0.01 to 50 kg/(h*kg), preferably in the range from 0.1 to 40 kg/(h*kg), further preferably in the range from 0.5 to 30 kg/(h*kg), the catalyst hourly space velocity being defined as the mass of stream S1 in kg per hour and per unit mass of aldol condensation catalyst in kg.
60. The process according to embodiment 58 or 59, wherein the contacting in (ii) in a fixed bed reactor is effected at an absolute pressure in the range from 0.5 to 5 bar, further preferably in the range from 0.8 to 3 bar, further preferably in the range from 1 to 1.8 bar.
61. The process according to any of embodiments 47 to 60, wherein stream S2 obtained in (ii) is at a temperature in the range from 200 to 450° C., preferably in the range from 250 to 400° C., further preferably in the range from 300 to 400° C.
62. The process according to any of embodiments 47 to 61, wherein the ratio of the volume of acrylic acid to the sum total of the volumes of formaldehyde and acetic acid in stream S2 obtained in (ii) is in the range from 0.1:1 to 2.0:1, preferably in the range from 0.4:1 to 1.2:1.

U.S. Provisional Patent Application No. 62/253,704, filed Nov. 11, 2015, is incorporated into the present application by literature reference. With regard to the abovementioned teachings, numerous changes and deviations from the present invention are possible. It can therefore be assumed that the invention, within the scope of the appended claims, can be performed differently from the way described specifically herein.

The present invention is illustrated in detail by the examples which follow.

### EXAMPLES

I. Analysis

1.1 Gas Chromatography
For analysis of the product stream, an online Agilent 7890A GCMS system was used.
Sampling was effected by a 10-port valve having a 500 μL sample loop or 1000 μL sample loop.
The analysis parameters can be expressed as follows:
MS/FID:
FFAP 25 m×0.32 mm×0.5 μm, carrier gas He, split 5:1, column flow rate 15 mL/min
TCD:
DB624 3 m×0.25 mm×1.4 μm
   Volamine 60 m×0.32 mm, carrier gas He, split 5:1, column flow rate 15 mL/min
TCD2:
RTX5 30 m×0.32 mm×1.0 μm
   Select permanent gases/CO2 HR carrier gas He, split 2:1, column flow rate: 30 mL/min
The temperature program was selected as follows:
hold at 40° C. for 2.5 min
heat to 105° C. at a heating rate of 20 K/min
heat to 225° C. at a heating rate of 40 K/min
hold at 225° C. for 2.75 min

II. Chemicals

| Chemical | Supplier | Purity |
|---|---|---|
| vanadium(V) oxide | Sigma Aldrich | >99.6% |
| oxalic acid dihydrate | Acros Organics | >99% |
| phosphoric acid | Acros Organics | >85% |
| ammonium metatungstate | Sigma Aldrich | >99% |
| tin(II) oxalate | Merck | >98% |
| potassium nitrate | Merck | >99% |
| lanthanum(III) nitrate hexahydrate | Sigma Aldrich | >99% |
| bismuth(III) nitrate pentahydrate | Sigma Aldrich | >99.99% |
| molybdenum oxide | Sigma Aldrich | >99.5 |

II. Preparation of Highly Concentrated Solutions of V₂O₅ in Aqueous Oxalic Acid

1.1 Molar Solution of V₂O₅ in Oxalic Acid

A 2 L three-neck flask was initially charged with 800 mL of aqueous oxalic acid dihydrate solution. While stirring, 1.1 mol of V₂O₅ were added to this solution and heated to 80° C. by means of a heating bath and refluxed. Oxalic acid dihydrate in solid form was then added in portions to the orange-brown suspension and the flask was sealed again. Evolution of gas and foam was observed here (redox reaction between V₂O₅ and oxalic acid). The addition of oxalic acid dihydrate was then repeated until the original suspension had become a deep blue solution. For this purpose, about three times the molar amount of oxalic acid dihydrate was needed (based on the molar amount of V₂O₅). The vanadium was present in the form of a solution of vanadyl oxalate VO(C₂O₄) with a molar concentration of vanadium of 2.2 mol/L. The solution thus obtained was cooled down to room temperature and transferred quantitatively into a 1 L standard flask (rinsing in with demineralized water, DM water). DM water (demineralized water) was used to make it up to 1 liter.

The mass of vanadium pentoxide to be weighed in (Sigma Aldrich Prod. No.: 221899) was determined by the following formula:

$$m ( V 2 O 5 ) = \frac{2 \cdot M ( V ) \cdot c \cdot V}{wt \%}$$

M(V)=molar mass of V c=concentration of the solution to be prepared
V=batch volume % by wt.=vanadium content of the V₂O₅ (manufacturer's certificate of analysis)

$$m ( V 2 O 5 ) = \frac{2 \cdot 50.94 g \text{/} mol \cdot 1.1 mol \text{/} L \cdot 1 L}{0.562} = 199.41 g$$

III Catalysts

III.1 General Details

The ignition loss (LOI hereinafter) of the support was determined beforehand. In this way, the exact content of oxidic components was known and it was possible to correct the starting support weight with this value. It was thus possible to ensure that the desired loading with active components was attained. The LOI of the Q20C support (CARiACT Q20C silica from Fuji Silysia) was 2.95%.

The impregnations were conducted to 100% of the water uptake (hereinafter 100% ICW) with mixed solutions of DM water and active component.

The loadings in the case of supported catalysts were given in “% by weight on support”. This means that, for example, for a “9.36V/11.3P/Q20C” catalyst, for the loading with vanadium, 9.36% by weight of the mass of support used had to be loaded onto the support as vanadium.

III.2 Preparation of the Catalysts of the Invention (IE)

31.18 g of the Q20C support were weighed into a porcelain dish (base diameter 18 cm) and placed onto an agitator. The latter was set such that the sample was kept in motion. By means of a 3 mL disposable pipette, the vanadium impregnation solution was applied dropwise uniformly to the support and homogenized with a spatula. The mixture then remained on the agitator for 30 minutes and was subsequently dried in an air circulation drying cabinet at 80° C. As soon as the sample was dried, it was cooled back down to room temperature.

Lastly, the sample was impregnated with the phosphorus impregnation solution (identical procedure) and likewise dried.

For the further elements, tungsten and optionally tin, it was to be noted that vanadium was preferably always impregnated as the first element and phosphorus as the last. Thus, if further elements were applied in addition to vanadium and phosphorus, vanadium was preferably always impregnated as the first element and then dried. Gradually, all the further elements were applied by this procedure. As the final impregnation, phosphorus was always applied as phosphoric acid solution.

It is also possible to conduct co-impregnations. For this purpose, impregnation solutions with several components were prepared and impregnated for the corresponding step.

After the final drying, the samples were calcined. For this purpose, they were heated to 260° C. in a muffle furnace (M110 from Heraeus) in an air stream (1 L/min) with a heating ramp of 1 K/min and kept at 260° C. for two hours, and then cooled down to room temperature. The samples were taken out of the muffle furnace and fine fractions formed (<315 μm) were removed by manual sieving.

Typically, all the components were used as aqueous solutions. Exceptions to this were tin(II) oxalate, which had good solubility only in semiconcentrated nitric acid (1 mol/L), and MoO₃, which was converted in 0.9 molar oxalic acid solution at 80° C. overnight. It was possible to dilute the solution formed to 2 mol/L with DM water.

Calculation Example

Support weight, LOI corrected

31.18 g−31.18 g*0.0295=30.260 g

Water uptake of the support (100% ICW)

31.18 g*1.04 mL/g=32.427 mL˜32.43 mL

Calculation of the mass of vanadium

m_{(V)}=(m₍ₛᵤₚₚₒᵣₜ₎−m₍ₛᵤₚₚₒᵣₜ₎*LOI)*% by wt. on support_{(V)}

m_{(V)}=(31.18 g−31.18 g*0.0295)*0.0936=2.832 g

Calculation of the volume of VO(C₂O₄) solution

m_{(V)}=M_{(V)}*c_{(V)}*V_{(V)}→V_{(V)}=m_{(V)}/(M_{(V)}*c_{(V)})

V_{(V)}=2.832 g/(50.94 g/mol*2.2 mol/L)=25.27 mL

Calculation of the mass of phosphorus

m_{(P)}=(m₍ₛᵤₚₚₒᵣₜ₎−m₍ₛᵤₚₚₒᵣₜ₎*LOI)*% by wt. on support_{(P)}

m_{(P)}=(31.18 g−31.18 g*0.0295)*0.113=3.419 g

Calculation of the volume of H₃PO₄ solution

m_{(P)}=M_{(P)}*c_{(P)}* V_{(P)}→V_{(P)}=m_{(P)}/(M_{(P)}*c_{(P)})

V_{(P)}=3.419 g/(30.97 g/mol*6 mol/L)=18.40 mL

Making up the impregnation solutions for 100% ICW
Vanadium impregnation solution

V(H2O content)=31.18 g*1.04 g/mL−V_{(V)}=35.43 mL−25.27 mL=10.16 mL
Phosphorus impregnation solution

V(H2O content)=31.18 g*1.04 g/mL−V_{(P)}=35.43 mL−18.40 mL=17.03 mL

111.3 Catalyst Compositions

Compositions of catalysts of the invention which have been prepared in III.2 are specified in tables 1 and 2 with percentages by weight and their molar proportions MMR of phosphorus (P), vanadium (V) and tungsten (W) or tin (Sn), and the molar ratio of phosphorus to the sum total of vanadium and tungsten and the molar ratio of vanadium to tungsten or the molar ratio of phosphorus to the sum total of vanadium, tungsten and tin. The molar proportion MMR of a component is defined as shown by way of example below for W:

$$MMR ( W ) = \frac{\frac{\% by wt . W}{M ( W )}}{\frac{\% by wt . W}{M ( W )} + \frac{\% by wt . V}{M ( V )} + \frac{\% by wt . P}{M ( P )}}$$

where M(W) is the molar mass of tungsten in g/mol, M(V) is the molar mass of vanadium in g/mol and M(P) is the molar mass of phosphorus in g/mol.

TABLE 1
Overview of catalysts of the invention comprising phosphorus (P), vanadium
(V) and tungsten (W) which have been used for catalytic studies

| | | | | | | | Molar ratio | |
|---|---|---|---|---|---|---|---|---|
| | | | | | | | of P to the | |
| | P [% | V [% | W [% | MMR | MMR | MMR | sum total | Molar ratio |
| Catalyst | by wt.] | by wt.] | by wt.] | (W) | (V) | (P) | of (V and W) | of V to W |
| IE1 | 12.43 | 6.56 | 10.14 | 0.090 | 0.22 | 0.69 | 2.23:1 | 2.44:1 |
| IE2 | 11.3 | 6.56 | 11.15 | 0.110 | 0.23 | 0.66 | 1.94:1 | 2.09:1 |
| IE3 | 12.43 | 6.56 | 11.15 | 0.100 | 0.22 | 0.68 | 2.13:1 | 2.20:1 |
| IE4 | 11.3 | 7.5 | 7.44 | 0.070 | 0.27 | 0.66 | 1.94:1 | 3.86:1 |
| IE5 | 11.3 | 7.5 | 8.12 | 0.080 | 0.26 | 0.66 | 1.94:1 | 3.25:1 |
| IE6 | 11.3 | 6.56 | 10.14 | 0.1 | 0.23 | 0.66 | 2.00:1 | 2.30:1 |
| IE7 | 12.42 | 7.5 | 11.61 | 0.1 | 0.24 | 0.66 | 1.94:1 | 2.40:1 |
| IE12 | 15.14 | 8.43 | 14.495 | 0.108 | 0.23 | 0.67 | 2.00:1 | 2.09:1 |
| IE13 | 12.42 | 6.56 | 11.15 | 0.1 | 0.22 | 0.68 | 2.13:1 | 2.20:1 |
| IE14 | 12.42 | 6.56 | 12.27 | 0.11 | 0.22 | 0.67 | 2.03:1 | 2.00:1 |
| IE17 | 11.3 | 7.5 | 6.76 | 0.07 | 0.27 | 0.66 | 1.94:1 | 3.86:1 |
| IE19 | 11.3 | 7.5 | 8.12 | 0.08 | 0.26 | 0.66 | 1.94:1 | 3.25:1 |
| IE20 | 12.43 | 6.56 | 11.15 | 0.10 | 0.22 | 0.68 | 2.12:1 | 2.12:1 |

TABLE 2
Overview of catalysts of the invention comprising phosphorus (P), vanadium
(V), tungsten (W) and tin (Sn) which have been used for catalytic studies

| | | | | | | | | | Molar ratio |
|---|---|---|---|---|---|---|---|---|---|
| | | | | | | | | | of P to the |
| | | | | | | | | | sum total |
| | Sn [% | P [% | V [% | W [% | MMR | MMR | MMR | MMR | of (V, W |
| Catalyst | by wt.] | by wt.] | by wt.] | by wt.] | (Sn) | (W) | (V) | (P) | and Sn) |
| IE8 | 2.18 | 12.43 | 8.43 | 0 | 0.03 | 0 | 0.28 | 0.69 | 2.2 |
| IE18 | 2.18 | 11.3 | 6.56 | 6.76 | 0.03 | 0.07 | 0.23 | 0.66 | 2 |
| IE9 | 1.09 | 11.3 | 8.43 | 1.69 | 0.02 | 0.02 | 0.3 | 0.66 | 1.9 |
| IE10 | 2.18 | 11.3 | 7.5 | 3.38 | 0.03 | 0.03 | 0.27 | 0.66 | 2 |
| IE11 | 4.37 | 11.3 | 6.56 | 3.38 | 0.07 | 0.03 | 0.23 | 0.66 | 2 |

Tables 3, 4 and 5 below indicate compositions of comparative catalysts which have been prepared according to III.2.

TABLE 3
Overview of comparative catalysts which have been used for catalytic studies

| | Bi [% | Mo [% | P [% | V [% | W [% | MMR | MMR | MMR | MMR | MMR |
|---|---|---|---|---|---|---|---|---|---|---|
| Catalyst | by wt.] | by wt.] | by wt.] | by wt.] | by wt.] | (Bi) | (Mo) | (W) | (V) | (P) |
| CE1 | 0 | 0 | 11.3 | 9.36 | 0 | 0 | 0 | 0 | 0.33 | 0.67 |
| CE2 | 0 | 0 | 12.43 | 9.36 | 0 | 0 | 0 | 0 | 0.31 | 0.69 |
| CE3 | 0 | 0 | 11.3 | 10.30 | 0 | 0 | 0 | 0 | 0.36 | 0.64 |
| CE4 | 7.68 | 0 | 11.3 | 7.5 | 0 | 0.07 | 0 | 0 | 0.27 | 0.66 |
| CE5 | 0 | 5.3 | 11.3 | 6.56 | 0 | 0 | 0.1 | 0 | 0.23 | 0.66 |
| CE6 | 0 | 3.5 | 11.3 | 7.5 | 0 | 0 | 0.07 | 0 | 0.27 | 0.67 |
| CE8 | 0 | 1.76 | 11.3 | 9.36 | 0 | 0 | 0.03 | 0 | 0.32 | 0.64 |
| CE10 | 7 | 0 | 12.7 | 11.4 | 3.8 | 0.03 | 0 | 0.33 | 0.05 | 0.60 |

TABLE 4
Overview of comparative catalysts comprising phosphorus (P), vanadium (V) and tungsten
(W), where the molar ratio of phosphorus to the sum total of vanadium and tungsten
is outside the inventive range, and which were used for catalytic studies

| | | | | | | | Molar ratio | |
|---|---|---|---|---|---|---|---|---|
| | | | | | | | of P to the | |
| | P [% | V [% | W [% | MMR | MMR | MMR | sum total | Molar ratio |
| Catalyst | by wt.] | by wt.] | by wt.] | (W) | (V) | #7 (P) | of (W and V) | of V to W |
| CE11 | 13.56 | 7.5 | 6.76 | 0.06 | 0.24 | 0.70 | 2.33 | 4.00 |
| CE12 | 13.56 | 6.56 | 10.14 | 0.09 | 0.21 | 0.70 | 2.33 | 2.33 |
| CE13 | 12.42 | 8.43 | 14.495 | 0.122 | 0.256 | 0.621 | 1.64 | 2.10 |
| CE14 | 13.51 | 8.43 | 14.495 | 0.129 | 0.272 | 0.599 | 1.49 | 2.11 |
| CE15 | 12.42 | 8.43 | 11.15 | 0.1 | 0.26 | 0.64 | 1.78 | 2.60 |
| CE16 | 11.3 | 7.5 | 11.61 | 0.11 | 0.26 | 0.63 | 1.70 | 2.36 |

TABLE 5
Overview of comparative catalysts comprising phosphorus (P), vanadium
(V) and tungsten (W), where the molar ratio of vanadium to tungsten is
outside the inventive range, and which were used for catalytic studies

| | | | | | | | Molar ratio | |
|---|---|---|---|---|---|---|---|---|
| | | | | | | | of P to the | |
| | P [% | V [% | W [% | MMR | MMR | MMR | sum total | Molar ratio |
| Catalyst | by wt.] | by wt.] | by wt.] | (W) | (V) | (P) | of (W and V) | of V to W |
| CE9 | 11.3 | 8.43 | 3.38 | 0.03 | 0.3 | 0.66 | 2.00 | 10.00 |
| CE17 | 12.43 | 8.43 | 3.38 | 0.03 | 0.28 | 0.69 | 2.23 | 9.33 |

III.4 Catalytic Studies/Use of the Catalysts in the Preparation of Acrylic Acid

The catalytic studies were conducted on pulverulent samples, for which a spall fraction having a particle size in the range from 0.315 to 0.5 mm was used. For preparation for the studies, the samples were positioned in tubular reactors between two inert particle beds consisting of quartz glass spall, the laden reactors were installed into the catalysis apparatus, a 16-tube high-throughput screening system, and the samples present therein were subjected to the test protocols.

For this purpose, a stream consisting of formaldehyde, acetic acid, water and argon was heated to 175° C. and hence evaporated. The gaseous mixture was then contacted with an aldol condensation catalyst according to the inventive examples (1E) and comparative examples (CE) in powder form at 1.1 bar [temperature and GHSV as specified in tables 6 to 22; GHSV=total volume flow rate of stream S1, in m³/h, per unit catalyst volume, in m³, under standard conditions (0° C. and absolute pressure 1.013 bar) in h⁻¹]. The temperature was measured at the start of the experimentation by means of a thermocouple in the isothermal zone of the reactor, i.e. of the catalyst bed, and corresponded to the temperature at which the reactions were conducted. The product stream was subsequently diluted with nitrogen, and the composition was determined by gas chromatography.

Tables 6 to 22 show the averaged result, with testing of the samples for 12 h. Catalytic results with inventive catalysts (1E) and comparative catalysts (CE) under different reaction conditions were compared. A negative influence was understood to mean a lowering of the acrylic acid selectivity (S(ACR) [%]), and/or an increase in the selectivity for COx (S(COx)) and/or lowering of the carbon conversion (C). A positive influence was understood to mean an increase in the acrylic acid selectivity (S(ACR) [%]), and/or a lowering of the selectivity for COx (S(COx)) and/or an increase in the carbon conversion (C).

The carbon conversion (C) was calculated by the following equation:

C=100*(NC^{P}ₛᵤₘ/(NC^{E}_{FA}+NC^{E}_{ACE}))

NC^{P}ₛᵤₘ=(NC^{E}_{FA}+NC^{E}_{ACE})−(NC^{P}_{FA}+NC^{P}_{ACE});
NC^{E}_{FA}=number of carbon atoms present in the stream in the form of a formaldehyde source;
NC^{E}_{ACE}=number of carbon atoms present in the stream in the form of acetic acid;
NC^{P}_{FA}=number of carbon atoms present in the product stream in the form of a formaldehyde source;
NC^{E}_{ACE}=number of carbon atoms present in the product stream in the form of acetic acid.
The acrylic acid selectivity (S) was calculated by the following formula:

S=100(NC^{P}_{AS}/NC^{P}ₛᵤₘ).

TABLE 6
Overview of acrylic acid selectivity (S(ACR)), selectivity
for COx (S(COx)) and carbon conversion (C) for inventive
catalysts (IE) compared to comparative catalysts (CE)

| Catalyst | S(COx) [%] | S(ACR) [%] | Carbon conversion (C) [%] |
|---|---|---|---|
| IE7 | 6.6 | 84.7 | 57.2 |
| CE1 | 8.6 | 79.8 | 54.3 |
| CE15 | 6.8 | 82.8 | 45.0 |
| CE1 | 8.1 | 81.7 | 44.4 |

Settings: T=350° C., GHSV [h⁻¹]=1000, acetic acid content=9% by volume, formaldehyde content=9% by volume, H₂O content=15.2% by volume, oxygen content=1.08% by volume

The inventive catalysts exhibited a positive influence on the selectivity of acrylic acid formation and a positive influence on the formation of COₓ.

TABLE 7
Overview of acrylic acid selectivity (S(ACR)), selectivity
for COx (S(COx)) and carbon conversion (C) for inventive
catalysts (IE) compared to comparative catalysts (CE)

| Catalyst | S(COx) [%] | S(ACR) [%] | Carbon conversion (C) [%] |
|---|---|---|---|
| IE14 | 5.9 | 86.8 | 47.1 |
| IE3 | 6.9 | 85.7 | 53.5 |
| IE13 | 5.5 | 86.7 | 41.5 |
| IE1 | 4.7 | 86.8 | 37.9 |
| CE2 | 10.9 | 81.3 | 53.2 |
| CE3 | 14.9 | 77.5 | 63.6 |
| CE1 | 15.1 | 77.6 | 61.9 |
| CE17 | 8.6 | 83.5 | 51.0 |
| CE15 | 12.7 | 80.2 | 67.0 |

Settings: T=370° C., GHSV [h⁻¹]=1256, acetic acid content=9% by volume, formaldehyde content=9% by volume, H₂O content=15.2% by volume, oxygen content=2.75% by volume

The inventive catalysts exhibited a positive influence on the selectivity of acrylic acid formation and a positive influence on the formation of COₓ.

TABLE 8
Overview of acrylic acid selectivity (S(ACR)), selectivity
for COx (S(COx)) and carbon conversion (C) for inventive
catalysts (IE) compared to comparative catalysts (CE)

| Catalyst | S(COx) [%] | S(ACR) [%] | Carbon conversion (C) [%] |
|---|---|---|---|
| IE2 | 4.8 | 87.3 | 50.5 |
| IE6 | 4.7 | 87.7 | 49.4 |
| IE20 | 7.6 | 84.7 | 58.2 |
| IE19 | 6.7 | 85.7 | 56.4 |
| IE4 | 6.6 | 85.5 | 56.0 |
| IE17 | 6.2 | 86.1 | 55.7 |
| CE1 | 8.8 | 81.7 | 58.0 |
| CE3 | 9.4 | 80.4 | 56.8 |
| CE1 | 9.5 | 80.9 | 57.4 |
| CE5 | 10.8 | 81.1 | 54.1 |
| CE16 | 8.1 | 83.7 | 60.0 |
| CE15 | 7.8 | 83.9 | 60.2 |

Settings: T=370° C., GHSV [h⁻¹]=1256, acetic acid content=9% by volume, formaldehyde content=9% by volume, H₂O content=15.2% by volume, oxygen content=1.38% by volume

The inventive catalysts exhibited a positive influence on the selectivity of acrylic acid formation and a positive influence on the formation of COₓ. The comparative catalysts which comprised bismuth exhibited a negative influence on the selectivity of acrylic acid formation and the carbon conversion (C).

TABLE 9
Overview of acrylic acid selectivity (S(ACR)), selectivity
for COx (S(COx)) and carbon conversion (C) for
inventive catalysts (IE) compared to comparative catalysts
(CE) which comprised bismuth (Bi)

| Catalyst | S(COx) [%] | S(ACR) [%] | Carbon conversion (C) [%] |
|---|---|---|---|
| IE3 | 6.9 | 85.7 | 53.5 |
| CE4 | 7.0 | 63.0 | 10.0 |

Settings: T=370° C., GHSV [h⁻¹]=1256, acetic acid content=9% by volume, formaldehyde content=9% by volume, H₂O content=15.2% by volume, oxygen content=2.75% by volume

The comparative catalyst which comprised bismuth exhibited a negative influence on the selectivity of acrylic acid formation and the carbon conversion.

TABLE 10
Overview of acrylic acid selectivity (S(ACR)), selectivity
for COx (S(COx)) and carbon conversion (C) for
inventive catalysts (IE) compared to comparative catalysts
(CE) which comprised molybdenum (Mo)

| Catalyst | S(COx) [%] | S(ACR) [%] | Carbon conversion (C) [%] |
|---|---|---|---|
| IE8 | 6.8 | 85.2 | 51.5 |
| CE6 | 10.1 | 82.0 | 55.5 |
| CE5 | 10.8 | 81.1 | 54.1 |
| CE8 | 10.0 | 81.0 | 55.2 |

Settings: T=370° C., GHSV [h⁻¹]=1256, acetic acid content=9% by volume, formaldehyde content=9% by volume, H₂O content=15.2% by volume, oxygen content=1.38% by volume

The comparison showed the negative influence of molybdenum on the selectivity of acrylic acid formation and the negative influence on the formation of COₓ.

TABLE 11
Overview of acrylic acid selectivity (S(ACR)), selectivity
for COx (S(COx)) and carbon conversion (C) for
inventive catalysts (IE) which comprised tungsten (W)
and tin (Sn) compared to comparative catalysts (CE)

| Catalyst | S(COx) [%] | S(ACR) [%] | Carbon conversion (C) [%] |
|---|---|---|---|
| IE18 | 4.4 | 87.6 | 45.4 |
| IE10 | 6.0 | 86.1 | 53.1 |
| IE11 | 4.3 | 87.3 | 42.3 |
| IE9 | 7.3 | 84.5 | 56.6 |
| CE1 | 9.5 | 80.9 | 57.4 |

Settings: T=370° C., GHSV [h⁻¹]=1256, acetic acid content=9% by volume, formaldehyde content=9% by volume, H₂O content=15.2% by volume, oxygen content=1.38% by volume

The comparison showed the positive influence of tin- and tungsten-containing inventive catalysts on the selectivity of acrylic acid formation and the positive influence on the formation of COₓ.

TABLE 12
Overview of acrylic acid selectivity (S(ACR)), selectivity
for COx (S(COx)) and carbon conversion (C) for inventive
catalysts (IE) compared to comparative catalysts (CE)

| Catalyst | S(COx) [%] | S(ACR) [%] | Carbon conversion (C) [%] |
|---|---|---|---|
| IE19 | 11.4 | 82.6 | 52.7 |
| IE7 | 12.3 | 81.2 | 57.4 |
| CE1 | 14.6 | 78.6 | 60.0 |

Settings: T=340° C., GHSV [h⁻¹]=1000, acetic acid content=9% by volume, formaldehyde content=9% by volume, H₂O content=15.2% by volume, oxygen content=1.944% by volume

The comparison showed the positive influence of vanadium- and tungsten-containing catalysts on the selectivity of acrylic acid formation and the positive influence on the formation of COₓ.

TABLE 13
Overview of acrylic acid selectivity (S(ACR)), selectivity
for COx (S(COx)) and carbon conversion (C) for inventive
catalysts (IE) compared to comparative catalysts (CE)

| Catalyst | S(COx) [%] | S(ACR) [%] | Carbon conversion (C) [%] |
|---|---|---|---|
| IE19 | 10.6 | 83.8 | 39.6 |
| IE5 | 10.1 | 84.8 | 44.6 |
| CE1 | 13.0 | 81.1 | 45.9 |

Settings: T=340° C., GHSV [h⁻¹]=3500, acetic acid content=9% by volume, formaldehyde content=9% by volume, H₂O content=15.2% by volume, oxygen content=1.944% by volume

The comparison showed the positive influence of vanadium- and tungsten-containing catalysts on the selectivity of acrylic acid formation and the positive influence on the formation of COₓ.

TABLE 14
Overview of acrylic acid selectivity (S(ACR)), selectivity
for COx (S(COx)) and carbon conversion (C) for inventive
catalysts (IE) compared to comparative catalysts (CE)

| Catalyst | S(COx) [%] | S(ACR) [%] | Carbon conversion (C) [%] |
|---|---|---|---|
| IE19 | 8.4 | 85.7 | 53.2 |
| IE5 | 8.4 | 85.9 | 60.4 |
| CE1 | 11.0 | 82.3 | 62.1 |

Settings: T=350° C., GHSV [h⁻¹]=1000, acetic acid content=9% by volume, formaldehyde content=9% by volume, H₂O content=15.2% by volume, oxygen content=1.944% by volume

The comparison showed the positive influence of vanadium- and tungsten-containing catalysts on the selectivity of acrylic acid formation and the positive influence on the formation of COₓ.

TABLE 15
Overview of acrylic acid selectivity (S(ACR)), selectivity
for COx (S(COx)) and carbon conversion (C) for inventive
catalysts (IE) compared to comparative catalysts (CE)

| Catalyst | S(COx) [%] | S(ACR) [%] | Carbon conversion (C) [%] |
|---|---|---|---|
| IE19 | 10.1 | 82.8 | 46.8 |
| IE5 | 9.3 | 83.9 | 50.3 |
| IE7 | 5.3 | 90.7 | 54.9 |
| CE1 | 12.9 | 79.6 | 51.8 |

Settings: T=350° C., GHSV [h⁻¹]=3500, acetic acid content=9% by volume, formaldehyde content=9% by volume, H₂O content=15.2% by volume, oxygen content=1.944% by volume

The comparison showed the positive influence of vanadium- and tungsten-containing catalysts on the selectivity of acrylic acid formation and the positive influence on the formation of COₓ.

TABLE 16
Overview of acrylic acid selectivity (S(ACR)), selectivity
for COx (S(COx)) and carbon conversion (C) for inventive
catalysts (IE) compared to comparative catalysts (CE)

| Catalyst | S(COx) [%] | S(ACR) [%] | Carbon conversion (C) [%] |
|---|---|---|---|
| IE19 | 12.8 | 81.5 | 58.8 |
| IE5 | 11.9 | 88.2 | 77.4 |
| CE1 | 14.4 | 76.5 | 68.6 |

Settings: T=350° C., GHSV [h⁻¹]=800, acetic acid content=9% by volume, formaldehyde content=9% by volume, H₂O content=15.2% by volume, oxygen content=1.944% by volume

The comparison showed the positive influence of vanadium- and tungsten-containing catalysts on the selectivity of acrylic acid formation and the positive influence on the formation of COₓ.

TABLE 17
Overview of acrylic acid selectivity (S(ACR)), selectivity
for COx (S(COx)) and carbon conversion (C) for inventive
catalysts (IE) compared to comparative catalysts (CE)

| Catalyst | S(COx) [%] | S(ACR) [%] | Carbon conversion (C) [%] |
|---|---|---|---|
| IE19 | 8.5 | 84.2 | 52.7 |
| IE5 | 8.8 | 84.1 | 57.2 |
| CE1 | 11.8 | 80.2 | 59.5 |

Settings: T=360° C., GHSV [h^{-1]}=3500, acetic acid content=9% by volume, formaldehyde content=9% by volume, H₂O content=15.2% by volume, oxygen content=1.944% by volume

The comparison showed the positive influence of vanadium- and tungsten-containing catalysts on the selectivity of acrylic acid formation and the positive influence on the formation of COₓ.

TABLE 18
Overview of acrylic acid selectivity (S(ACR)), selectivity
for COx (S(COx)) and carbon conversion (C) for inventive
catalysts (IE) compared to comparative catalysts (CE)

| Catalyst | S(COx) [%] | S(ACR) [%] | Carbon conversion (C) [%] |
|---|---|---|---|
| IE2 | 7.0 | 85.2 | 54.8 |
| IE6 | 5.5 | 86.6 | 47.8 |
| IE19 | 7.7 | 84.8 | 54.7 |
| IE4 | 8.7 | 83.3 | 55.9 |
| IE17 | 7.4 | 84.5 | 52.4 |
| CE1 | 11.6 | 80.0 | 56.3 |
| CE5 | 14.3 | 77.7 | 52.2 |
| CE16 | 9.8 | 82.0 | 62.9 |
| CE15 | 9.9 | 81.9 | 62.1 |

Settings: T=370° C., GHSV [h⁻¹]=1256, acetic acid content=13.5% by volume, formaldehyde content=13.5% by volume, H₂O content=22.8% by volume, oxygen content=2.75% by volume

The comparison showed the positive influence of vanadium- and tungsten-containing catalysts on the selectivity of acrylic acid formation and the positive influence on the formation of COₓ.

TABLE 19
Overview of acrylic acid selectivity (S(ACR)), selectivity
for COx (S(COx)) and carbon conversion (C) for inventive
catalysts (IE) compared to comparative catalysts (CE)

| Catalyst | S(COx) [%] | S(ACR) [%] | Carbon conversion (C) [%] |
|---|---|---|---|
| IE2 | 9.0 | 83.6 | 55.3 |
| IE6 | 7.2 | 85.6 | 49.3 |
| IE4 | 10.8 | 81.7 | 55.5 |
| IE17 | 9.7 | 83.2 | 53.2 |
| CE1 | 15.2 | 76.7 | 62.7 |
| CE1 | 15.9 | 76.8 | 57.9 |
| CE3 | 16.2 | 75.2 | 58.3 |
| CE1 | 14.4 | 78.0 | 56.5 |
| CE15 | 13.4 | 79.7 | 62.3 |
| CE16 | 13.1 | 80.0 | 63.5 |

Settings: T=370° C., GHSV [h⁻¹]=1256, acetic acid content=9% by volume, formaldehyde content=13.5% by volume, H₂O content=22.8% by volume, oxygen content=2.75% by volume

The comparison showed the positive influence of vanadium- and tungsten-containing catalysts on the selectivity of acrylic acid formation and the positive influence on the formation of COₓ.

TABLE 20
Overview of acrylic acid selectivity (S(ACR)),
selectivity for COx (S(COx)) and carbon conversion (C) for
inventive catalysts (IE) compared to comparative catalysts (CE)

| | | | Carbon |
|---|---|---|---|
| | | | conversion (C) |
| Catalyst | S(COx) [%] | S(ACR) [%] | [%] |
| IE4 | 9.5 | 81.6 | 56.0 |
| IE2 | 9.6 | 81.5 | 55.8 |
| IE6 | 8.1 | 83.1 | 53.3 |
| IE17 | 10.0 | 81.1 | 55.6 |
| CE9 | 11.6 | 79.5 | 54.3 |
| CE16 | 12.2 | 78.3 | 57.4 |
| CE15 | 12.2 | 78.4 | 57.1 |
| CE1 | 13.1 | 76.2 | 55.5 |
| CE3 | 13.6 | 75.1 | 55.0 |

Settings: T=370° C., GHSV [h^{-1]}=1256, acetic acid content=13.5% by volume, formaldehyde content=9% by volume, H₂O content=15.2% by volume, oxygen content=2.75% by volume

The comparison showed the positive influence of vanadium- and tungsten-containing catalysts on the selectivity of acrylic acid formation and the positive influence on the formation of COₓ.

TABLE 21
Overview of acrylic acid selectivity (S(ACR)),
selectivity for COx (S(COx)) and carbon conversion (C) for
inventive catalysts (IE) compared to comparative catalysts (CE)

| | | | Carbon conversion (C) |
|---|---|---|---|
| | S(COx) [%] | S(ACR) [%] | [%] |
| IE12 | 7.5 | 85.3 | 54.4 |
| CE13 | 13.0 | 80.0 | 68.2 |
| CE14 | 10.2 | 82.8 | 62.7 |

Settings: T=370° C., GHSV [h^{-1]}=1256, acetic acid content=13.5% by volume, formaldehyde content=9% by volume, H₂O content=15.2% by volume, oxygen content=2.75% by volume

The comparison showed the positive influence of vanadium- and tungsten-containing catalysts on the selectivity of acrylic acid formation and the positive influence on the formation of COₓ.

TABLE 22
Overview of acrylic acid selectivity (S(ACR)),
selectivity for COx (S(COx)) and carbon conversion (C) for
inventive catalysts (IE) compared to comparative catalysts (CE)

| | | | Carbon conversion (C) |
|---|---|---|---|
| | S(COx) [%] | S(ACR) [%] | [%] |
| CE11 | 5 | 85.6 | 32.9 |
| CE12 | 4 | 86 | 30.5 |
| IE20 | 6.8 | 85.7 | 53.5 |

Settings: T=370° C., GHSV [h⁻¹]=1256, acetic acid content=13.5% by volume, formaldehyde content=9% by volume, H₂O content=15.2% by volume, oxygen content=2.75% by volume

The comparison showed the positive influence of the inventive molar ratio of P to the sum total of (W and V) on the carbon conversion (C).

1. An oxidic composition, comprising; vanadium, tungsten, phosphorus, oxygen and optionally tin, wherein the molar ratio of phosphorus to the sum total of vanadium, tungsten and any tin in the oxidic composition is in the range from 1.4:1 to 2.4:1.

2. The oxidic composition according to claim 1, wherein the molar ratio of phosphorus to the sum total of vanadium, tungsten and any tin is in the range from 1.8:1 to 2.3:1.

3. The oxidic composition according to claim 1, wherein the molar ratio of vanadium to tungsten in the oxidic composition is in the range from 10:1 to 1:100.

4. The oxidic composition according to claim 1, wherein the oxidic composition comprises not more than 1000 molar ppm, of molybdenum.

5. The oxidic composition according to claim 1, wherein the oxidic composition comprises not more than 1000 molar ppm, of bismuth.

6. The oxidic composition according to claim 1, wherein the oxidic composition comprises not more than 1000 molar ppm, of titanium.

7. The oxidic composition according to claim 1, wherein the oxidic composition comprises tin.

8. The oxidic composition according to claim 7, wherein the molar ratio of vanadium to tin in the oxidic composition is in the range from 100:1 to 1:100.

9. The oxidic composition according to claim 1, further comprising a support material.

10. The oxidic composition according to claim 1, wherein the oxidic composition is a catalyst.

11. The oxidic composition according to claim 1, wherein the oxidic composition is an unsupported catalyst.

12. The oxidic composition according to claim 9, wherein the oxidic composition is a supported catalyst.

13. A process for producing an oxidic composition, comprising:
providing a support material;
providing an aqueous vanadium solution, an aqueous tungsten solution, an aqueous phosphorus solution and optionally an aqueous tin solution;
impregnating the support material with the aqueous vanadium solution and the aqueous tungsten solution and optionally the aqueous tin solution;
optionally drying the resulting impregnated material;
impregnating the optionally dried material with the aqueous phosphorus solution;
optionally drying the resulting impregnated material;
calcining the optionally dried material.

14. The process according to claim 13 for producing an oxidic composition; said composition comprising vanadium, tungsten, phosphorus, oxygen and optionally tin, wherein the molar ratio of phosphorus to the sum total of vanadium, tungsten and any tin in the oxidic composition is in the range from 1.4:1 to 2.4:1.

15. The process according to claim 13, wherein the aqueous solutions provided comprise a total of not more than 1000 molar ppm, of molybdenum, not more than 1000 molar ppm, of bismuth and not more than 1000 molar ppm, of titanium.

16. The process according to claim 13, wherein the aqueous vanadium solution comprises vanadium citrate or vanadium oxalate or a mixture thereof, the aqueous phosphorus solution comprises phosphoric acid, the aqueous tin solution comprises tin oxalate, optionally as a mixture with nitric acid, and the aqueous tungsten solution comprises ammonium metatungstate.

17. The process according to claim 13, comprising
(i) providing the support material;
(ii) providing the aqueous vanadium solution, the aqueous tungsten solution; the aqueous phosphorus solution;
(iii) impregnating the support material with the aqueous vanadium solution;
(iv) optionally drying the material obtained in (iii);
(V) impregnating the material obtained in (iv) with the aqueous tungsten solution;
(vi) optionally drying the material obtained in (V);
(vii) impregnating the material obtained in (vi) with the aqueous phosphorus solution;
(viii) optionally drying the material obtained in (vii);
(ix) calcining the material obtained in (viii).

18. The process according to claim 17, wherein (ii) additionally comprises the providing of an aqueous tin solution and the process additionally comprises
(a) impregnating the material obtained in (iv) or that obtained in (vii) with the aqueous tin solution;
(b) optionally drying the material obtained in (a),
where (a) to (b) optionally follow (iv) and precede (V) or follow (vi) and precede (vii).

19. An oxidic composition, obtained or obtainable by a process according to claim 13.

20. A process for preparing acrylic acid from acetic acid and formaldehyde, comprising
(i) providing a stream S1 comprising acetic acid and formaldehyde;
(ii )contacting stream S1 with an aldol condensation catalyst comprising,
preferably consisting of, an oxidic composition according to claim 1 to obtain a stream S2 comprising acrylic acid.
